(19) United States
(12) Patent Application Publication
(10) Pub. No.: US 2007/0270959 A1
(43) Pub. Date: Nov. 22, 2007

(54) Arthroplasty device

(75) Inventor: Jean Dubousset, Paris (FR)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/405,802

(22) Filed: Apr. 18, 2006

(51) Int. Cl. A61F 2/44

(52) U.S. Cl. 623/17.11; 606/61

(57) ABSTRACT

An artificial vertebral joint having an anterior motion preserving system connected to a posterior motion preserving system is disclosed. The anterior motion preserving system may be permanently connected or selectively connected to the posterior motion preserving system. Further, the anterior and posterior motion preserving systems may include flexible members, such as springs, to preserve motion while providing stability.

### TECHNICAL FIELD

Embodiments of the invention relate generally to devices and methods for accomplishing spinal surgery, and more particularly in some embodiments, to spinal arthroplasty devices capable of being placed posteriorly into the vertebral disc space. Various implementations of the invention are envisioned, including use in total spine arthroplasty to replace, via a posterior approach, both the disc and facet functions of a natural spinal joint.

### BACKGROUND

In some exemplary aspects, the devices disclosed herein may include one or more features disclosed in the following prior patent applications, incorporated herein in their entirety by reference:
U.S. Utility patent application Ser. No. 11/031,602, filed on Jan. 7, 2005 and entitled “Spinal Arthroplasty Device and Method;”
U.S. Utility patent application Ser. No. 11/031,603, filed on Jan. 7, 2005 and entitled “Dual Articulating Spinal Device and Method;”
U.S. Utility patent application Ser. No. 11/031,780, filed on Jan. 7, 2005 and entitled “Split Spinal Device and Method;”
U.S. Utility patent application Ser. No. 11/031,904, filed on Jan. 7, 2005 and entitled “Interconnected Spinal Device and Method;”
U.S. Utility patent application Ser. No. 11/031,700, filed on Jan. 7, 2005 and entitled “Support Structure Device and Method;”
U.S. Utility patent application Ser. No. 11/031,783, filed on Jan. 7, 2005 and entitled “Mobile Bearing Spinal Device and Method;”
U.S. Utility patent application Ser. No. 11/031,781, filed on Jan. 7, 2005 and entitled “Centrally Articulating Spinal Device and Method;” and
U.S. Utility patent application Ser. No. 11/031,903, filed on Jan. 7, 2005 and entitled “Posterior Spinal Device and Method.”

As is known in the art, in the human anatomy, the spine is a generally flexible column that can take tensile and compressive loads, allows bending motion, and provides a place of attachment for ribs, muscles, and ligaments. The spine includes a plurality of vertebrae, including cervical, lumbar, and thoracic, and intervertebral discs formed there between. Each intervertebral disc is designed to absorb the compressive and tensile loads to which the spinal column may be subjected while simultaneously allowing adjacent vertebral bodies to move relative to each other a limited amount, particularly during bending (flexure) of the spine. Thus, the intervertebral discs are under constant muscular and/or gravitational pressure and generally are the first parts of the spine to show signs of “wear and tear”.

Adjacent vertebrae are also connected by a facet joint. Facet joint degeneration is also common because the facet joints are in almost constant motion with the spine. In fact, facet joint degeneration and disc degeneration frequently occur together. Typically, one is the primary problem while the other is a secondary problem resulting from the altered mechanics of the spine. Often by the time surgical options are considered, both facet joint degeneration and disc degeneration have occurred. For example, the altered mechanics of the facet joints and/or intervertebral disc may cause spinal stenosis, degenerative spondylolisthesis, and degenerative scoliosis. Disease, degradation, and trauma of the spine can lead to these and other conditions that require treatment to maintain, stabilize, or reconstruct the vertebral column. As the standard of care in spinal treatment moves from arthrodesis to arthroplasty, preserving motion and limiting further degradation in a spinal joint or in a series of spinal joints becomes increasingly more complex.

To date, standard treatments of the vertebral column have not adequately addressed the need for devices, systems, and procedures to treat joint degradation. Likewise, current techniques do not adequately address the impact that a treatment may have on the adjacent bone, soft tissue, or joint behavior. For example, stand-alone anterior spinal motion devices (or dynamic stabilization devices) do not fully stabilize the spine; they permit motion while resisting anterior-column load. For this reason, interbody motion devices are sometimes ineffective when there is any posterior muscular, ligamentus, or other instability. On the other hand, posterior dynamic stabilization devices do not substantially resist loads through the anterior column, nor can they provide anterior distraction. Thus, while both anterior and posterior dynamic devices permit motion, each is capable of providing something the other cannot.

Accordingly, there is a need for improved spinal arthroplasty devices that avoid the drawbacks and disadvantages of the known implants and surgical techniques.

### SUMMARY

In one embodiment, an artificial vertebral joint having an anterior motion preserving system connected to a posterior motion preserving system is provided. The anterior motion preserving system may be permanently connected or selectively connected to the posterior motion preserving system.

In a second embodiment, a prosthetic device for placement at least partially between a superior vertebra and a inferior vertebra is provided. The prosthetic device includes comprising an anterior motion preserving system and a posterior motion preserving system. The anterior motion preserving system includes a superior component with a superior surface for engaging a portion of the superior vertebra, an inferior component with an inferior surface for engaging a portion of the inferior vertebra, and a first linking member. The first linking member is connected to the superior component by a first flexible member. The first linking member is connected to the inferior component by a second flexible member. The posterior motion preserving system is connected to the anterior motion preserving system. The posterior motion preserving system includes an upper portion, a lower portion, and a second linking member. The second linking member is connected to the upper portion by a third flexible member. The second linking member is also connected to the lower portion by a fourth flexible member.

In a third embodiment, an artificial vertebral joint that includes a first prosthetic device and a second prosthetic device is provided. The first and second prosthetic devices each having an anterior motion preserving system connected to a posterior motion preserving system.

Additional and alternative features, advantages, uses, and embodiments are set forth in or will be apparent from the following description, drawings, and claims.

### BRIEF DESCRIPTION OF THE DRAWINGS

FIG. 1 is a perspective view of a portion of an intervertebral disc disposed between two adjacent vertebrae.

FIG. 2 is a side exploded elevation view of a portion of the spine showing a prosthetic device positioned between adjacent vertebrae according to one embodiment of the present invention.

FIG. 3 is an anterior elevation view of a portion of a lumbar spine showing two assembled prosthetic devices according to one embodiment of the present invention positioned between adjacent vertebrae.

FIG. 4 is a side elevation view of one of the prosthetic devices and adjacent vertebrae shown in FIG. 3.

FIG. 5 is a partial cross-sectional bottom-up-view of the assembled prosthetic devices of FIG. 3 and the adjacent vertebra.

FIG. 6 is a perspective posterior view of the assembled artificial prosthetic devices and adjacent vertebrae further including elastic posterior members.

### DESCRIPTION

The present disclosure relates generally to vertebral reconstructive devices, and more particularly, to devices and procedures for spinal arthroplasty. For the purposes of promoting an understanding of the principles of the invention, reference will now be made to the embodiments, or examples, illustrated in the drawings and specific language will be used to describe the embodiments. It will nevertheless be understood that no limitation of the scope of the invention is intended. Any alterations and further modifications of the described embodiments, and any further applications of the principles of the invention as described herein are contemplated as would normally occur to one skilled in the art to which the invention relates.

In particular, the drawings illustrate various embodiments of an artificial intervertebral joint for replacing the functions of an intervertebral disc or the combination of an intervertebral disc and at least one corresponding facet joint. Various embodiments of the artificial intervertebral joint according to the principles of the disclosure may be used for treating any of the problems that lend themselves to joint replacement including, for example and without limitation, degenerative changes of the spine, post-traumatic, discogenic, facet pain, or spondylolisthesis. Joint replacement may also be utilized to restore and/or maintain motion in multiple levels of the spine.

Disc degeneration may lead to disc collapse or loss of disc height, resulting in pain or neurodeficit. Similarly, degeneration of the facet joints may lead to pain or neurodeficit. When treating one degenerated region of the motion segment, the impact of the treatment on the surrounding regions should be considered. For example, inappropriate restoration of disc height to only a posterior portion of the interbody space may result in hyperkyphosis with loss of height in the anterior interbody area and placement of the anterior annulus in compression. Also, improvements to the anterior interbody area alone is difficult to achieve when instability such as spondylolisthesis or retrolisthesis exists. Likewise, appropriate restoration of disc height to only an anterior portion of the interbody space may result in hyperlordosis with loss of posterior disc height and compression of the posterior annulus and facet joints.

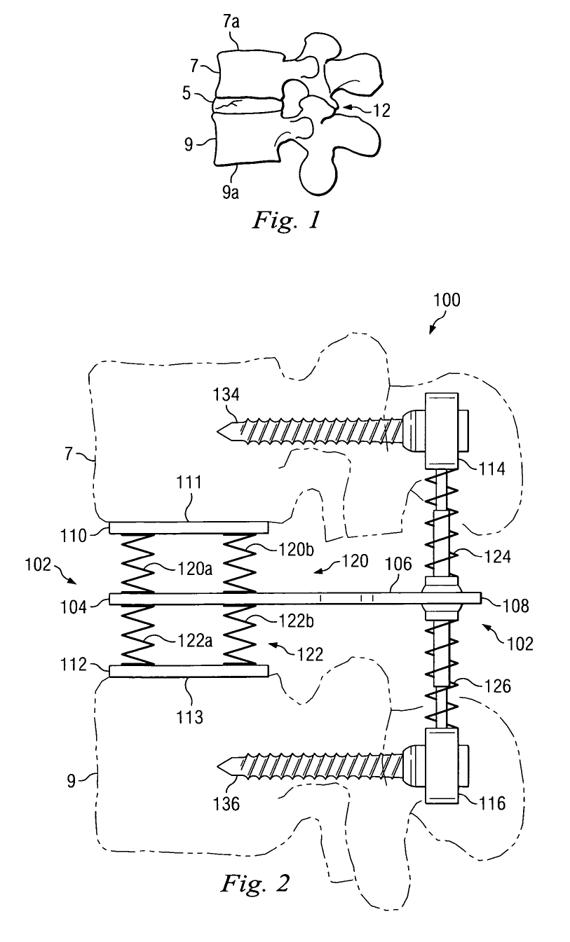

FIG. 1 shows a vertebral joint section or a “motion segment” 10 of a vertebral column. The motion segment includes a superior vertebra 7, an inferior vertebra 9, and a intervertebral disc 5. Each vertebra 7, 9 has a vertebral body 7a, 9a, respectively. As used herein, the term motion segment describes the overall relative movement between adjacent vertebrae 7, 9. This includes movement at the disc space, at the facet joints 12, and movement allowed through various tissue, ligaments and muscle compositions. Treatment, stabilization, or reconstruction of the vertebral motion segment 10 may be diagnosed and carried out in a systematic manner. To achieve an improved clinical outcome and a stable result, both an anterior region 12 and a posterior region 14 of the motion segment 10 may be treated simultaneously. This may be accomplished utilizing an anterior motion preservation system connected to a posterior motion preservation system, as described below.

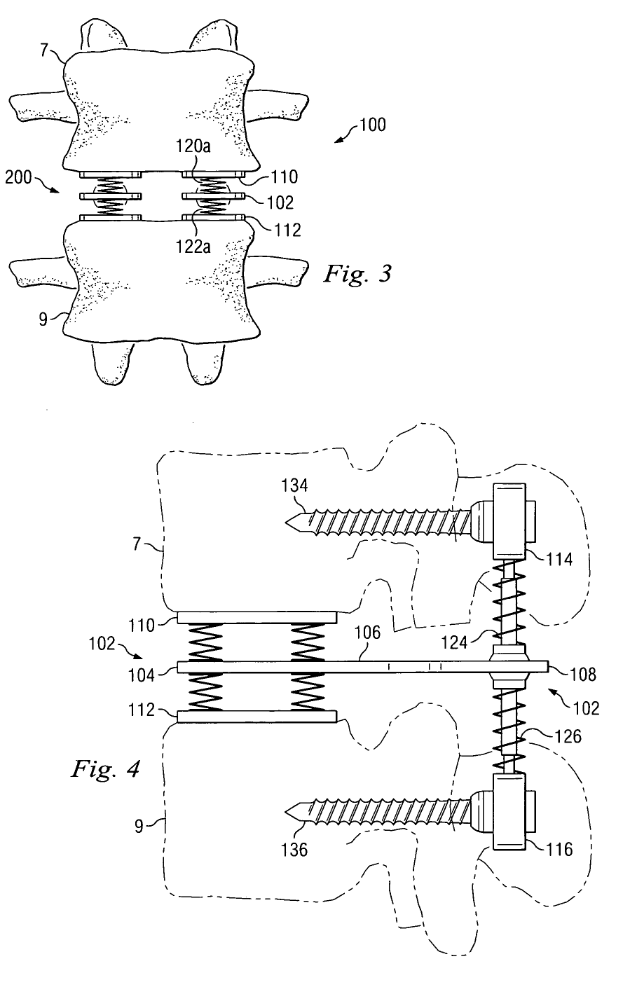

Referring now to FIGS. 2-5, shown therein is a first exemplary embodiment of an artificial intervertebral joint according to the present invention. As illustrated in FIGS. 3, 5, and 6, each joint is composed of pair of bilateral assemblies or arthroplasty devices 100, 200. One of the arthroplasty devices 100 is adapted for placement substantially on the left portion of the disc space, and the other arthroplasty device 200 is adapted for placement substantially on the right portion. The arthroplasty devices 100, 200 may be substantially identical such that either device may be placed on either side of the disc space. It is fully contemplated, however, that each device 100, 200 may be shaped or otherwise configured for placement on a particular side of the disc space. For clarity, further description will now be made primarily with respect to arthroplasty device 100. It is to be understood that arthroplasty device 200 may be substantially similar or identical to arthroplasty device 100 and, therefore, will not be described in further detail.

As shown in the exploded view of FIG. 2, arthroplasty device 100 includes both an anterior and a posterior motion preserving system. The anterior system includes an upper or superior component 110, a lower or inferior component 112, a linking member 102, a superior flexible linking component 120, and an inferior flexible linking component 122. The posterior system includes the linking member 102, a superior component 114, an inferior component 116, a superior flexible linking component 124, an inferior flexible linking component 126, a superior fixation device 134, and an inferior fixation device 136.

As shown and described, the linking member 102 extends from the anterior system of the arthroplasty device 100 to the posterior system of the arthroplasty device. The linking member 102 includes an anterior component 104, a posterior component 108, and a bridge component 106 extending between the anterior and posterior components. The linking member 102 serves to connect the anterior system to the posterior system.

While the linking member 102 is shown as a single component, it is fully contemplated that anterior component 104, bridge component 106, and posterior component 108 may be separate components adapted to be selectively connected or attached to each other. Having separate components allows selective attachment of the anterior motion preserving system to the posterior motion preserving system. In this regard, it is contemplated that the bridge component 106 may be incorporated into either the anterior or posterior component 104, 108 to create a two-piece linking member 102 instead of a three-piece linking member. Attachment of the components in these embodiments may be accomplished using any fastening mechanism known in the art including, for example, a threaded connection, a bolted connection, or a latched connection.

Utilizing separate components for the linking member 102 facilitates implantation of one motion preserving system at a time and possibly simplifies the surgical procedure. For example, implanting the arthroplasty device 100 could be either a three step implantation process—where the bridge 106 is a separate component—or a two step implantation process—where the bridge is incorporated into the anterior component 104 or posterior component 106. From a posterior approach the anterior motion preserving system could be implanted, then the bridge could be implanted and attached to anterior motion preserving system, then the posterior motion preserving system could be implanted and attached to the bridge, thereby connecting the anterior and posterior systems without having to implant both systems simultaneously. Further, it should be understood by one of ordinary skill in the art that the arthroplasty device could be implanted via an anterior approach or a combined anterior and posterior approach, although the advantages of a posterior procedure would be limited in such cases. For example, the anterior motion preserving system may be inserted from an anterior approach while the bridge and posterior motion preserving system are inserted posteriorly.

The linking member 102, the superior components 110, 114, and the inferior components 112, 116 of the anterior and posterior motion preserving systems may be formed of any suitable biocompatible material including metals such as cobalt-chromium alloys, titanium alloys, nickel titanium alloys, or stainless steel alloys. Ceramic materials such as aluminum oxide or alumina, zirconium oxide or zirconia, compact of particulate diamond, or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); or cross-linked UHMWPE. The anterior, posterior, and bridge components of the linking member 102; the superior components 110, 114; and the inferior components 112, 116 may each be formed of different materials, permitting metal on metal, metal on ceramic, metal on polymer, ceramic on ceramic, ceramic on polymer, or polymer on polymer constructions.

Referring now to the anterior motion preserving system, the superior component 110 includes engagement surface 111 adapted to engage an anterior surface of the superior vertebra 7. The inferior component 112 includes engagement surface 113 adapted to engage a superior surface of the inferior vertebra 9. The engagement surfaces 111, 113 may include features or coatings that enhance the fixation of the superior and inferior components 110, 112 to the superior and inferior vertebra 7, 9, respectively. For example, the surfaces 111, 113 may be roughened by chemical etching, bead-blasting, sanding, grinding, serrating, or diamond-cutting. All or a portion of the engagement surfaces 111, 113 of the superior and inferior components 110, 112 may also be coated with a biocompatible and osteoconductive material such as hydroxyapatite (HA), tricalcium phosphate (TCP), or calcium carbonate to promote bone ingrowth and fixation. Alternatively, osteoinductive coatings, such as proteins from the transforming growth factor (TGF) beta superfamily or bone-morphogenic proteins, such as BMP2 or BMP7, may be used. Other suitable features may include spikes, keels, ridges, or other surface textures designed to encourage fixation between the superior and inferior components 110, 112 and the vertebrae 7, 9.

The superior component 110 and the inferior component 112 are connected to the anterior component 104 of the linking member 102 via linking components 120, 122, respectively. The superior linking component 120 is shown as being comprised of springs 120a, 120b. Similarly, the inferior linking component 122 is shown as being comprised of springs 122a, 122b. Though the linking components 120, 122 are shown having two springs it is fully contemplated that the linking components may utilize a single spring or a plurality of springs. Further, linking components 120, 122 need not have springs at all. Linking components 120, 122 serve at least two purposes: connecting the superior and inferior components to the linking member 102 and preserving at least a limited amount of motion between vertebrae 7, 9 while supporting compressive and tensile loads on the anterior portion of the motion segment. Thus, it is contemplated that the linking components 120, 122, though shown as coiled springs, may be any device or feature adapted to provide cushioning or dampening as well as load bearing resistance or support. The linking components 120, 122 may be loaded in compression or tension depending upon the patient's condition. Further, though not shown it is fully contemplated that a flexible housing or sheath may be utilized to protect the structure and preserve the functioning of the linking components 120, 122 individually and the anterior motion preserving system as a whole.

Finally, the size and shape of the anterior system, including the linking member 102 and its bridge component 106, may be limited by the constraints of a posterior surgical approach. For example, the superior and inferior components 110, 112 may be configured to cover a maximum vertebral endplate area to dissipate loads and reduce subsidence while still fitting through the posterior surgical exposure, Kambin's triangle, and other neural elements.

Referring now to the posterior motion preserving system, the superior component 114 and the inferior component 116 of the posterior system are adapted to be secured to the vertebrae 7, 9 by fixation components 134, 136, respectively. In this regard, the superior and inferior components 114, 116 may include an aperture, opening, or other feature to facilitate attachment via the fixation components 134, 136. Fixation components 134, 136 are shown as pedicle screws. However, it is contemplated that the fixation components 134, 136 may be staples, adhesives, or other means of securing the superior and inferior components 114, 116 of the posterior motion preserving system.

The superior and inferior components 114, 116 may be shaped or oriented to permit the fixation components 134, 136 to be inserted into the vertebral bodies 7a, 9a. As shown best in FIG. 5, in this embodiment the pedicle screw passes through a wall of the vertebral body and achieves strong cortical fixation. In all embodiments, the fixation components 134, 136 may be recessed or otherwise configured so as not to interfere with articulations, soft tissues, and neural structures. In an alternative embodiment, the superior and inferior components 114, 116 may be shaped or oriented to permit the fixation components 134, 136 to be inserted extrapedicularly such that the fixation component travels a path angled or skewed away from a central axis defined through a pedicle. Extrapedicular fixation may be any fixation into the pedicle that does not follow a path down a central axis defined generally posterior-anterior through the pedicle. It is contemplated, though not preferred, that the fixation component engage the pedicle along the central axis of the pedicle.

Similar to the anterior motion preserving system, the superior component 114 and the inferior component 116 of the posterior motion preserving system are connected to the posterior component 108 of the linking member 102 via linking components 124, 126, respectively. The linking components 124, 126 are shown as being springs. The linking components 124, 126 are adapted to flex and preserve motion while supporting the loads on the posterior system. The linking components 124, 126 may allow some axial movement of the motion segment in addition to extension and flexion. In this regard, in addition to replacing the function of the facet joint the posterior motion preserving system may be used to supplement or replace the function of the posterior ligaments that limit mobility between adjacent vertebrae. Though the linking components 124, 126 are shown as springs it is fully contemplated that the linking components may be any device adapted to preserve motion and support loads in posterior region of the motion segment. Thus, it is contemplated that the linking components 120, 122, though shown as coiled springs, may include devices or features adapted to provide cushioning or dampening as well as load bearing resistance or support. Again the linking components 124, 126 may be loaded in compression or tension depending upon the patient's condition. Further, though not shown it is fully contemplated that a flexible housing or sheath may be utilized to protect the structure and preserve the functioning of the linking components 124, 126 individually and the anterior motion preserving system as a whole.

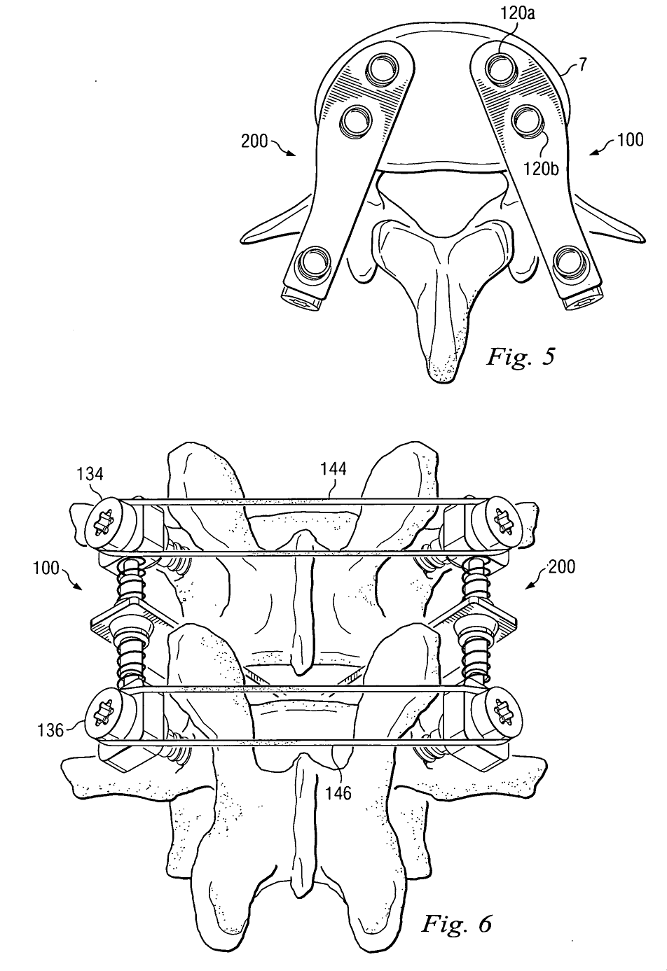

FIG. 6 shows an alternative embodiment of an artificial intervertebral joint according to the present invention. The interveterbal joint of FIG. 6 includes posterior tension bands 144, 146 and may utilize a arthroplasty devices substantially similar to arthroplasty devices 100, 200 described above. The posterior tension bands 144, 146 serve to prevent expansion and undesired movement. The posterior tension bands 144, 146 may be utilized by wrapping them around the corresponding pedicle screws or other convenient attachment points of the arthroplasty devices 100, 200 or the vertebrae 7, 9.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A prosthetic device for placement at least partially between a superior vertebra and an inferior vertebra, comprising:
an anterior motion preserving system having a superior component with a superior surface for engaging a portion of the superior vertebra, an inferior component with an inferior surface for engaging a portion of the inferior vertebra, and a first linking member connected to the superior component by a first flexible member, the first linking member connected to the inferior component by a second flexible member; and
a posterior motion preserving system connected to the anterior motion preserving system, the posterior motion preserving system having an upper portion, a lower portion, and a second linking member connected to the upper portion by a third flexible member, the second linking member connected to the lower portion by a fourth flexible member.

2. The prosthetic device of claim 1, wherein the first linking member and the second linking member are connected.

3. The prosthetic device of claim 2, wherein the first linking member and the second linking member are a single member.

4. The prosthetic device of claim 1, wherein the superior component is made of nitinol.

5. The prosthetic device of claim 4, wherein the inferior component is made of nitinol.

6. The prosthetic device of claim 1, wherein at least one of the first flexible member, the second flexible member, the third flexible member, or the fourth flexible member is a spring.

7. The prosthetic device of claim 1, wherein at least one of the first flexible member, the second flexible member, the third flexible member, or the fourth flexible member is a plurality of springs.

8. The prosthetic device of claim 1, wherein the posterior motion preserving system is adapted to replace a facet joint.

9. The prosthetic device of claim 1, wherein the upper portion is adapted to be secured to at least a portion of the superior vertebra.

10. The prosthetic device of claim 9, wherein the upper portion is adapted to be secured by a pedicle screw.

11. The prosthetic device of claim 10, wherein the lower portion is adapted to be secured to at least a portion of the inferior vertebra.

12. The prosthetic device of claim 11, wherein the lower portion is adapted to be secured by a pedicle screw.

13. The prosthetic device of claim 1, wherein the superior surface is configured to enhance fixation to the superior vertebra.

14. The prosthetic device of claim 13, wherein the inferior surface is configured to enhance fixation to the inferior vertebra.

15. The prosthetic device of claim 14, wherein at least one of the superior surface and the inferior surface includes a keel.

16. The prosthetic device of claim 14, wherein at least one of the superior surface and the inferior surface is treated with an osteoconductive material.

17. The prosthetic device of claim 14, wherein at least one of the superior surface and the inferior surface is treated with an osteoinductive material.

18. A prosthetic device for placement at least partially between a superior vertebra and an inferior vertebra, comprising:
an anterior motion preserving system; and
a posterior motion preserving system connected to the anterior motion preserving system, the posterior motion preserving system including an upper portion, a lower portion, and a first linking member connected to the upper portion by a first flexible member, the first linking member connected to the lower portion by a second flexible member.

19. The prosthetic device of claim 18, wherein the first flexible member is a spring.

20. The prosthetic device of claim 19, wherein the second flexible member is a spring.

21. The prosthetic device of claim 18, wherein the linking member connects the posterior motion preserving system to the anterior motion preserving system.

22. The prosthetic device of claim 21, wherein the anterior motion preserving system includes a superior component adapted to engage a portion of the superior vertebra, an inferior component adapted to engage a portion of the inferior vertebra, and a second linking member connected to the superior component and connected to the inferior component.

23. The prosthetic device of claim 22, wherein the second linking member is connected to the superior component via a third flexible member.

24. The prosthetic device of claim 23, wherein the second linking member is connected to the inferior component via a fourth flexible member.

25. An artificial vertebral joint for placement at least partially between a superior vertebra and an inferior vertebra, comprising:
a first prosthetic device including a first anterior motion preserving system connected to a first posterior motion preserving system via a first linking member; the first anterior motion preserving system having a first superior component, a first inferior component, the first linking member connected to the first superior component by a first spring, and the first linking member connected to the first inferior component by a second spring; the first posterior motion preserving system having a first upper portion, a first lower portion, the first linking member connected to the first upper portion by a third spring, and the first linking member connected to the first lower portion by a fourth spring; and
a second prosthetic device including a second anterior motion preserving system connected to a second posterior motion preserving system via a second linking member; the second anterior motion preserving system having a second superior component, a second inferior component, the second linking member connected to the second superior component by a fifth spring, and the second linking member connected to the second inferior component by a sixth spring; the second posterior motion preserving system having a second upper portion, a second lower portion, the second linking member connected to the second upper portion by a seventh spring, and the second linking member connected to the second lower portion by an eighth spring.
